(19) United States
(12) Patent Application Publication
(10) Pub. No.: US 2023/0223130 A1
(43) Pub. Date: Jul. 13, 2023

(54) NON-TRANSITORY COMPUTER-READABLE STORAGE MEDIUM, INFORMATION PROCESSING APPARATUS, AND INFORMATION PROCESSING METHOD

(71) Applicant: JAPAN COMPUTER VISION CORP., Tokyo (JP)

(72) Inventors: Toshihiro UTSUMI, Tokyo (JP); Chikashi OKAMOTO, Tokyo (JP); Masamichi KATAGIRI, Tokyo (JP)

(73) Assignee: JAPAN COMPUTER VISION CORP., Tokyo (JP)

(21) Appl. No.: 17/859,910

(22) Filed: Jul. 7, 2022

(30) Foreign Application Priority Data
Jan. 7, 2022 (JP) 2022-001677

(51) Int. Cl. G16H 20/60; G16H 20/30; G06V 40/10

(52) U.S. Cl. CPC G16H 20/60; G16H 20/30; G06V 40/10

(57) ABSTRACT

A non-transitory computer-readable recording medium with an information processing program stored thereon, wherein the program instructs a computer to execute an acquisition step of acquiring current body information that is information on a body of a target user who is a processing target user at a present time and future body information that is information on a body that the target user wants to have after a lapse of a predetermined time since the present time, an estimation step of estimating recommended food information on a food recommended to be taken by the target user among foods that are captured in a meal image obtained by imaging a meal, on the basis of the current body information and the future body information acquired at the acquiring, and a providing step of providing the recommended food information estimated at the estimating to the target user.

### CROSS-REFERENCE TO RELATED APPLICATION(S)

The present application claims priority to and incorporates by reference the entire contents of Japanese Patent Application No. 2022-001677 filed in Japan on Jan. 7, 2022.

### BACKGROUND OF THE INVENTION

### 1. Field of the Invention

The present invention relates to an information processing program, an information processing apparatus, and an information processing method.

### 2. Description of the Related Art

Conventionally, various technologies for supporting health management of a user are known. For example, an image or a video of how a target person has a meal is captured by an imaging apparatus, and meal record information including a plurality of items related to the meal is acquired and stored form the captured image or video. Further, biological data of the target person is measured by a measurement apparatus, and biological data information on the measured biological data is stored. Furthermore, a technology for generating relevance data by analyzing a relevance between each of the items in the meal record information and variation in the biological data on the basis of the meal record information and the biological data information that are stored, generating an advice about meals on the basis of the generated relevance data, and providing the generated advice to the target person is known.
Patent Literature 1: Japanese Laid-open Patent Publication No. 2017-54163

However, in the conventional technology as described above, only the advice that is about meals and that is generated based on the past meal record information and the biological data information on the target person is provided to a user, so that it is not always possible to appropriately support health management of the user.

### SUMMARY OF THE INVENTION

It is an object of the present invention to at least partially solve the problems in the conventional technology.

According to one aspect of an embodiment, a non-transitory computer-readable recording medium with an information processing program stored thereon, wherein the program instructs a computer to execute an acquisition step of acquiring current body information that is information on a body of a target user who is a processing target user at a present time and future body information that is information on a body that the target user wants to have after a lapse of a predetermined time since the present time, an estimation step of estimating recommended food information on a food recommended to be taken by the target user among foods that are captured in a meal image obtained by imaging a meal, on the basis of the current body information and the future body information acquired at the acquiring, and a providing step of providing the recommended food information estimated at the estimating to the target user.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram illustrating a configuration example of an information processing apparatus according to one embodiment;

FIG. 2 is a diagram for explaining an acceptance process of accepting input of current body information and future body information according to one embodiment;

FIG. 3 is a diagram for explaining a process of estimating recommended food information according to one embodiment;

FIG. 4 is a diagram for explaining a process of estimating recommended exercise information according to one embodiment;

FIG. 5 is a flowchart illustrating the flow of information processing according to one embodiment; and

FIG. 6 is a hardware configuration diagram illustrating an example of a computer that implements functions of the information processing apparatus.

### DETAILED DESCRIPTION OF THE PREFERRED EMBODIMENTS

Modes (hereinafter, referred to as “embodiments”) for carrying out an information processing program, an information processing apparatus, and an information processing method according to the present application will be described in detail below with reference to the drawings. The information processing program, the information processing apparatus, and the information processing method according to the present application are not limited by the embodiments below. Further, in each of the embodiments described below, the same components are denoted by the same reference symbols, and repeated explanation will be omitted.

### Embodiment

1. Configuration of Information Processing Apparatus

An information processing apparatus 100 is a terminal apparatus that is owned and used by a user who uses a health management service for supporting health management of the user. The information processing apparatus 100 may be a mobile terminal, such as a smartphone or a tablet personal computer (PC), or may be a notebook PC or a desktop PC.

The information processing apparatus 100 provides an advice about meals or exercises that are needed to bring body information on a user closer to body information desired by the user, on the basis of body information on a body shape or the like of the user at the present time and body information on a body shape or the like that the user wants to have in the future. For example, the information processing apparatus 100 provides the user with recommended food information on a food that is recommended to be taken and provides the user with non-recommended food information on a food that is not recommended to be taken, among foods that are captured in a meal image obtained by capturing an image of a meal.

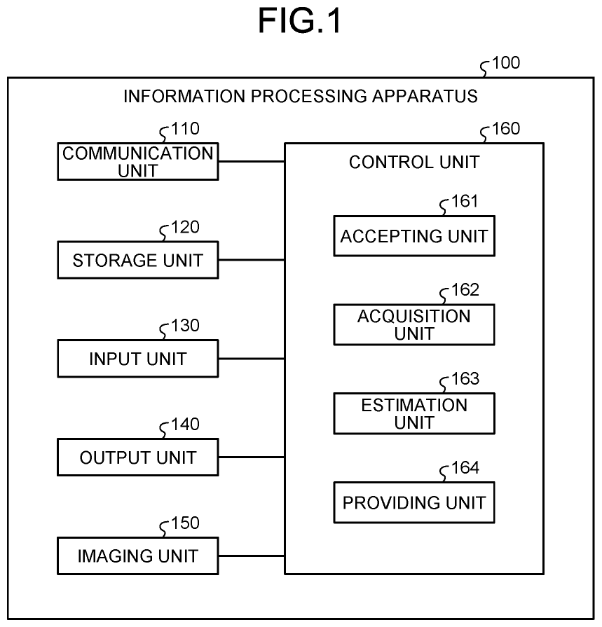

FIG. 1 is a diagram illustrating a configuration example of the information processing apparatus 100 according to the embodiment. The information processing apparatus 100 includes a communication unit 110, a storage unit 120, an input unit 130, an output unit 140, an imaging unit 150, and a control unit 160.

Communication Unit 110

The communication unit 110 is implemented by, for example, a network interface card (NIC) or the like. Further, the communication unit 110 is connected to a network in a wired or wireless manner, and transmits and receives information to and from a server apparatus that is managed by a service provider who provides a health management service, for example.

Storage Unit 120

The storage unit 120 is implemented by, for example, a semiconductor memory device, such as a random access memory (RAM) or a flash memory, or a storage apparatus, such as a hard disk or an optical disk. Specifically, the storage unit 120 stores therein various programs (one example of an information processing program), such as an application related to the health management service.

Input Unit 130

The input unit 130 receives input of various kinds of operation from the user. For example, the input unit 130 may receive various kinds of operation from the user via a display screen (for example, the output unit 140) with a touch panel function. Further, the input unit 130 may receive various kinds of operation from a button that is arranged on the information processing apparatus 100 or a keyboard or a mouse that is connected to the information processing apparatus 100. For example, the input unit 130 receives editing operation on an image.

Output Unit 140

The output unit 140 is, for example, a display screen that is implemented by a liquid crystal display, an organic electro-luminescence (EL) display, or the like, and is a display apparatus for displaying various kinds of information. The output unit 140 displays various kinds of information under the control of the control unit 160. For example, the output unit 140 displays an image that is accepted by an accepting unit 161. Meanwhile, if a touch panel is adopted in the information processing apparatus 100, the input unit 130 and the output unit 140 are integrated. Further, in the following description, the output unit 140 may be described as a screen.

Imaging Unit 150

The imaging unit 150 implements a camera function for imaging a target object. The imaging unit 150 includes, for example, an optical system, such as a lens, and an imaging device, such as a charge coupled device (CCD) image sensor or a complementary metal oxide semiconductor (CMOS) sensor. Specifically, the imaging unit 150 captures an image in accordance with operation performed by the user. For example, the imaging unit 150 captures a user image in which at least a part of a body of the user is captured. Further, the imaging unit 150 captures a meal image in which a meal is captured.

Control Unit 160

The control unit 160 is a controller and is implemented by causing a central processing unit (CPU), a micro processing unit (MPU), or the like to execute various programs (corresponding to one example of the information processing program) stored in a storage apparatus inside the information processing apparatus 100 by using a random access memory (RAM) as a work area, for example. Further, the control unit 160 is a controller and implemented by an integrated circuit, such as an application specific integrated circuit (ASIC) or a field programmable gate array (FPGA).

The control unit 160 includes, as functional units, the accepting unit 161, an acquisition unit 162, an estimation unit 163, and a providing unit 164, and may implement or execute operation of information processing to be described below. Meanwhile, an internal configuration of the control unit 160 is not limited to the configuration as illustrated in FIG. 1, and a different configuration may be adopted as long as it is possible to perform the information processing to be described later. Furthermore, each of the functional units represents functions of the control unit 160 and need not always be physically separated.

Accepting Unit 161

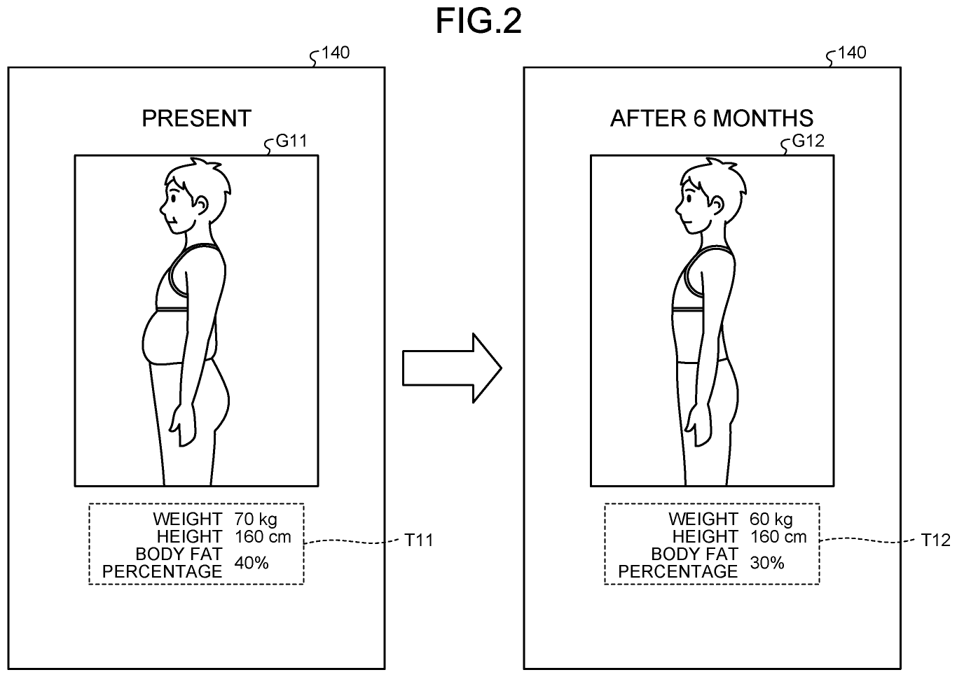

FIG. 2 is a diagram for explaining an acceptance process of accepting input of current body information and future body information according to the embodiment. The accepting unit 161 accepts, from a target user who is a processing target user, the current body information that is information on a body of the target user at the present time. For example, the accepting unit 161 accepts, as one example of the current body information, a target user image G11 in which a body shape of a lateral half of the body of the target user is captured. For example, the accepting unit 161 accepts the target user image G11 that is captured by the imaging unit 150. Further, the accepting unit 161 accepts, from the target user via the input unit 130, input of each of values T11 representing current weight, height, and a body fat percentage of the target user. The accepting unit 161 accepts, as one example of the current body information, each of the values T11 that represent current weight, current height, and a current body fat percentage of the target user that are input by the target user. Subsequently, upon accepting the current body information, the accepting unit 161 stores the accepted current body information in the storage unit 120 in association with information on acceptance date and time of the current body information. Furthermore, the output unit 140 displays, on a screen, the target user image G11 and each of the values T11 of the weight, the height, and the body fat percentage of the target user accepted by the accepting unit 161.

Moreover, the accepting unit 161 accepts, from the target user, the future body information that is information on a body of the target user that the target user wants to have after a lapse of a predetermined time since the present time. The accepting unit 161 may accept setting of a period corresponding to the predetermined time from the target user. The future body information is, in other words, body information on a body shape, weight, or the like as a future goal of the target user. In the example illustrated in FIG. 2, the accepting unit 161 accepts, from the target user, information indicating a body shape as a goal that the target user wants to achieve after a lapse of six months since the present time. For example, the accepting unit 161 accepts, from the target user via the input unit 130, editing operation on a body shape of the target user that is captured in the target user image G11 that has already been accepted. For example, the accepting unit 161 accepts editing operation of thinning an abdomen, reducing a facial contour, or thinning an arm or a leg. Subsequently, the accepting unit 161 accepts, as one example of the future body information, a target user image G12 that has been edited through the editing operation that is accepted from the target user. Further, the accepting unit 161 accepts, from the target user via the input unit 130, input of each of values T12 representing weight, height, and a body fat percentage of the target user as goals that the target user will achieve after a lapse of six months since the present time. The accepting unit 161 accepts, as one example of the future body information, input of each of the values T12 that represent the weight, the height, and the body fat percentage of the target user and that are input by the target user. Upon accepting the future body information, the accepting unit 161 stores the accepted future body information in the storage unit 120 in association with information on acceptance date and time of the future body information. Further, the output unit 140 displays, on a screen, the edited target user image G12 accepted by the accepting unit 161 and each of the values T12 representing the weight, the height, and the body fat percentage of the target user. Meanwhile, the accepting unit 161 may estimate each of the values T12 representing the weight, the height, and the body fat percentage of the target user corresponding to a target body shape, on the basis of each of the values T11 representing the weight, the height, and the body fat percentage and the edited target user image G12, which have already been accepted.

Meanwhile, the case has been illustrated in FIG. 2 in which the accepting unit 161 accepts, as one example of the current body information, the target user image G11 in which the body shape of the lateral half of the body of the target user is captured, but the accepting unit 161 may accept any target user image as long as at least a part of the body of the target user is captured in the target user image. For example, the accepting unit 161 accepts a target user image in which at least any of the whole body, an upper half of the body, a lower half of the body, a face, an arm, a leg, a chest, an abdomen, a buttocks, and a back of the target user is captured. Further, the accepting unit 161 may accept a target user image in which the target user is captured in an arbitrary direction. For example, the accepting unit 161 may accept a target user image in which the target user is captured in any of a front direction, a lateral direction, or a back direction.

Furthermore, while the case is illustrated in FIG. 2 in which the accepting unit 161 accepts, as one example of the current body information and the future body information, each of the values that represent the weight, the height, and the body fat percentage of the target user and that are input by the target user, the accepting unit 161 may accept any kind of information as long as the information represents the body information on the target user. For example, the accepting unit 161 may accept each of values representing weight, chest circumference, waist circumference, hip circumference, a body mass index (BMI), a body fat percentage, muscle mass, a basal metabolic rate, estimated bone quantity of the target user, or each of values representing a body fat percentage or muscle mass for each of body parts of the target user. Moreover, the accepting unit 161 may accept not only a body shape of the whole body of the target user, but also an image in which a body shape of a specific part of the body (for example, a shape of the face, a shape of the arm, a shape of the leg, or the like) of the target user. Furthermore, the accepting unit 161 may accept editing operation on not only the whole body of the target user, but also the body shape of a specific part of the body.

Moreover, while the case is illustrated in FIG. 2 in which the accepting unit 161 accepts, from the target user, the editing operation of thinning the abdomen, reducing the facial contour, or thinning the arm or the leg of the target user that is captured in the target user image G11, the accepting unit 161 may accept, from the target user, different editing operation on the body shape of the target user. Specifically, the accepting unit 161 may accept, from the target user, editing operation of editing the body shape of the target user captured in the target user image G11 into a muscular shape. For example, the accepting unit 161 may accept, from the target user, editing operation of making each of muscles, such as a biceps, a deltoid muscle, an abdominal muscle, or a greater pectoral muscle, of the target user captured in the target user image G11 bigger, or editing operation of designing each of the muscles in a favorable manner.

Furthermore, the accepting unit 161 accepts a meal image from the target user. For example, the accepting unit 161 accepts a meal image that is captured by the imaging unit 150. For example, the accepting unit 161 accepts a meal image in which a plurality of foods are captured. Here, the foods may be food ingredients or cooked foods that are obtained by cooking food ingredients.

Acquisition Unit 162

The acquisition unit 162 acquires the current body information that is information on a body of the target user at the present time, and the future body information that is information on a body that the target user wants to have after a lapse of the predetermined time since the present time. The acquisition unit 162 acquires the current body information and the future body information that are accepted by the accepting unit 161. Specifically, when the accepting unit 161 accepts the meal image, the acquisition unit 162 acquires the current body information and the future body information on the target user. More specifically, when the accepting unit 161 accepts the meal image, the acquisition unit 162 refers to the storage unit 120 and acquires the current body information and the future body information on the target user.

Estimation Unit 163

The estimation unit 163 estimates the recommended food information on a food that is recommended to be taken by the target user among the foods that are captured in the meal image obtained by imaging the meal, on the basis of the current body information and the future body information acquired by the acquisition unit 162. Here, the recommended food information may include the non-recommended food information on a food that is not recommended to be taken by the target user. In other words, the estimation unit 163 may estimate only one of the recommended food information and the non-recommended food information, or may estimate both of the recommended food information and the non-recommended food information.

Specifically, when the accepting unit 161 accepts the meal image, the estimation unit 163 refers to the storage unit 120 and acquires the current body information and the future body information on the target user. Subsequently, the estimation unit 163 estimates an amount of each of nutrients that need to be taken by the target user in a set period of time, on the basis of the acquired current body information and the acquired future body information on the target user. Here, the amount of each of the nutrients that need to be taken by the target user includes calories of foods in addition to an amount of each of nutrients, such as lipid, carbohydrate, protein, vitamin, and mineral. For example, the estimation unit 163 estimates calories that need to be taken by the target user in the set period of time, on the base of a difference between the current weight and a future goal weight of the target user. Further, for example, the estimation unit 163 estimates an amount of fat that needs to be taken by the target user in the set period of time, on the basis of a difference between the current body fat percentage and a future goal body fat percentage of the target user. Furthermore, for example, the estimation unit 163 estimates an amount of protein that needs to be taken by the target user in the set period of time, on the basis of a difference between the current muscle mass and future goal muscle mass of the target user.

Subsequently, the estimation unit 163 estimates an amount of each of the nutrients that need to be taken by the target user in a day in the set period of time, on the basis of the amounts of the nutrients that need to be taken by the target user in the set period of time. For example, the estimation unit 163 estimates calories that need to be taken by the target user in a day in the set period of time by dividing the calories that need to be taken by the target user in the set period of time by days included in the set period of time. Furthermore, for example, the estimation unit 163 estimates an amount of fat that needs to be taken by the target user in a day in the set period of time by dividing the amount of fat that needs to be taken by the target user in the set period of time by the days included in the set period of time. Moreover, for example, the estimation unit 163 estimates an amount of protein that needs to be taken by the target user in a day in the set period of time by dividing the amount of protein that needs to be taken by the target user in the set period of time by the days included in the set period of time. Subsequently, the estimation unit 163 estimates the recommended food information on a food that is recommended to be taken by the target user among the foods that are captured in the meal image obtained by imaging the meal, on the basis of each of the nutrients that need to be taken by the target user in a day.

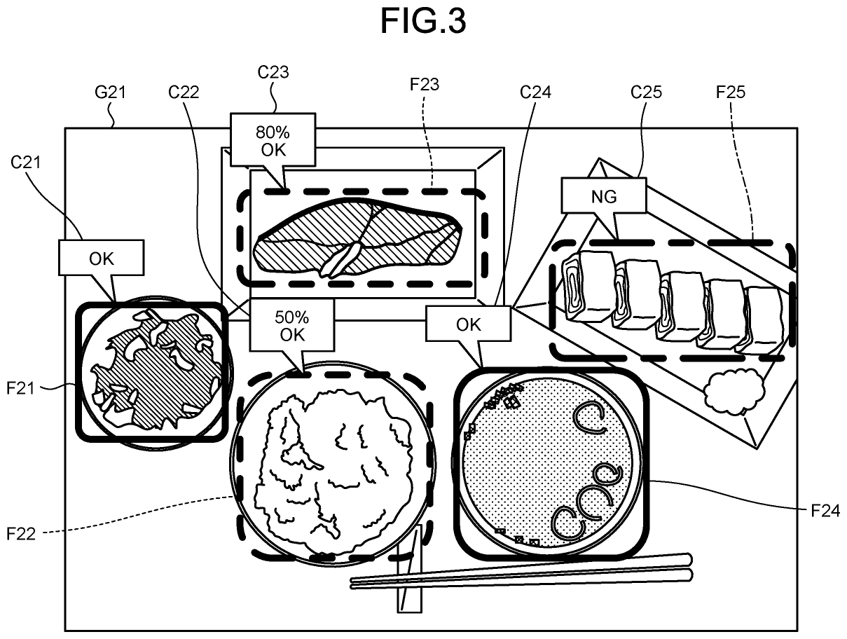

FIG. 3 is a diagram for explaining a process of estimating the recommended food information according to the embodiment. In FIG. 3, the accepting unit 161 accepts a meal image G21 in which five foods F21 to F25 are captured. When the accepting unit 161 accepts the meal image G21, the estimation unit 163 acquires the meal image G21 from the accepting unit 161. Subsequently, the estimation unit 163 estimates an amount of each of nutrients included in each of the foods captured in the meal image G21. For example, if the meal image is input, the estimation unit 163 estimates the amount of each of the nutrients included in each of the foods captured in the meal image G21 by using a machine learning model M1 that is trained to output the amount of each of the nutrients included in each of the foods captured in the meal image. Here, the amounts of the nutrients included in the foods include the amount of each of the nutrients, such as lipid, carbohydrate, protein, vitamin, and mineral, and calories of the foods.

Subsequently, after estimating the amount of each of the nutrients included in each of the foods, the estimation unit 163 estimates a recommended food that is a food recommended to be taken by the target user, on the basis of the estimated amount of each of the nutrients included in each of the foods. For example, the estimation unit 163 estimates the amount of each of the nutrients in all of the five foods F21 to F25 by adding, for each of the nutrients, the estimated amounts of each of the nutrients in the five foods F21 to F25. Subsequently, the estimation unit 163 identifies the recommended food on the basis of a comparison between the amount of each of the nutrients in all of the five foods F21 to F25 and the amount of each of the nutrients that need to be taken by the target user in a day. For example, if the amount of each of the nutrients in all of the five foods F21 to F25 is smaller than the amount of each of the nutrients that need to be taken by the target user in a day, the estimation unit 163 may identify all of the five foods F21 to F25 as the recommended foods. In contrast, if the amount of each of the nutrients in all of the five foods F21 to F25 is larger than the amount of each of the nutrients that need to be taken by the target user in a day, the estimation unit 163 identifies a combination of foods for which the amount of each of the nutrients becomes equal to or smaller than the amount of each of the nutrients that need to be taken by the target user in a day among combinations of foods selected from among the five foods F21 to F25. In the example illustrated in FIG. 3, the estimation unit 163 identifies a combination of the foods F21 to F24 as the combination of foods for which the amount of each of the nutrients becomes equal to or smaller than the amount of each of the nutrients that need to be taken by the target user in a day among the combinations of the foods selected from among the five foods F21 to F25. Subsequently, after identifying the combination of the foods for which the amount of each of the nutrients becomes equal to or smaller than the amount of each of the nutrients that need to be taken by the target user in a day, the estimation unit 163 identifies the foods related to the identified combination as the recommended foods. In the example illustrated in FIG. 3, the estimation unit 163 identifies the foods F21 to F24 related to the identified combination as the recommended foods. If the recommended foods are identified, the estimation unit 163 displays information that allows the recommended foods to be visually recognized on the screen. For example, the estimation unit 163 displays characters C21 to C24 of “OK” indicating the recommended foods at positions located within predetermined ranges from positons of frames enclosing the foods F21 to F24 that are identified as the recommended foods.

Further, if the recommended foods are identified, the estimation unit 163 estimates a recommended intake amount that is an intake amount of the recommended food that is recommended to be taken by the target user. For example, the estimation unit 163 estimates a nutrient whose intake needs to be reduced by the target user in the set period of time, on the basis of the acquired current body information and the acquired future body information on the target user. For example, if a difference in weight exceeds a first threshold based on a difference between the current weight and the future goal weight of the target user, the estimation unit 163 identifies carbohydrate as a nutrient whose intake needs to be reduced by the target user. Furthermore, for example, if a difference in the body fat percentage exceeds a second threshold based on a difference between the current body fat percentage and the future goal body fat percentage of the target user, the estimation unit 163 identifies fat as a nutrient whose intake needs to be reduced by the target user.

Subsequently, if the nutrient whose intake needs to be reduced by the target user is identified, the estimation unit 163 determines whether a food that contains the nutrient whose intake needs to be reduced by the target user and whose amount is equal to or larger than a predetermined value is present among the foods that are identified as the recommended foods. In the example illustrated in FIG. 3, the estimation unit 163 identifies carbohydrate as the nutrient whose intake needs to be reduced by the target user. Subsequently, the estimation unit 163 determines that the food F22 that contains carbohydrate whose amount is equal to or larger than a third threshold is present among the foods F21 to F24 that are identified as the recommended foods. If the estimation unit 163 determines that the food F22 that contains carbohydrate whose amount is equal to or larger than the third threshold is present, the estimation unit 163 estimates an intake amount of the food F22 by which the intake amount of carbohydrate becomes smaller than the third threshold. For example, the estimation unit 163 estimates a half amount as the intake amount of the food F22 by which the intake amount of carbohydrate becomes smaller than the third threshold. The estimation unit 163 identifies the intake amount of the food F22 by which the intake amount of carbohydrate becomes smaller than the third threshold as the recommended intake amount. Subsequently, if the estimation unit 163 estimates the recommended intake amount, the estimation unit 163 displays information that allows the recommended intake amount to be visually recognized on the screen. For example, the estimation unit 163 displays a character C22 of “50% OK” indicating that the recommended intake amount is a half at a position located within a predetermined range from the position of the frame enclosing the food F22. The estimation unit 163 similarly displays a character C23 of “80% OK” indicating that the recommended intake amount is 80 percent at a position located within a predetermined range from the position of the frame enclosing the food F23.

In contrast, if a food that is not identified as the recommended food is present, the estimation unit 163 identifies the food that is not identified as the recommended food as a non-recommended food as a food that is not recommended to be taken by the target user. In the example illustrated in FIG. 3, the estimation unit 163 identifies, as the non-recommended food, the food F25 that is not identified as the recommended food. If the estimation unit 163 identifies the non-recommended food, the estimation unit 163 displays information that allows the non-recommended food to be visually recognized on the screen. For example, the estimation unit 163 displays a character C25 of “NG” indicating the non-recommended food at a position located within a predetermined range from a position of a frame enclosing the food F25.

While the case has been illustrated in FIG. 3 in which the estimation unit 163 estimates a nutrient whose intake needs to be reduced by the target user in the set period of time, the estimation unit 163 may estimate a nutrient that needs to be positively taken by the target user in the set period of time on the basis of the acquired current body information and the acquired future body information on the target user. For example, if a difference in muscle mass exceeds a fourth threshold based on a difference between the current muscle mass and the future goal muscle mass of the target user, the estimation unit 163 estimates protein as a nutrient that needs to be positively taken by the target user.

Further, while the case has been illustrated in FIG. 3 in which when the meal image is input, the estimation unit 163 estimates the amount of each of the nutrients included in each of the foods captured in the meal image G21 by using the machine learning model M1 that is trained to output the amount of each of the nutrients included in each of the foods captured in the meal image, but a method of estimating the amounts of the nutrients included in the foods by the estimation unit 163 is not limited to this example. For example, the estimation unit 163 detects each of the foods captured in the meal image G21 by using a well-known object recognition technique, and identifies types of the detected foods. Subsequently, the estimation unit 163 acquires information indicating the amount of each of nutrients included in the identified foods. For example, the information processing apparatus 100 acquires, in advance, food nutrition information in which an amount of each of nutrients included in a food and a type of the food are associated, and stores the food nutrition information in the storage unit 120. The estimation unit 163 refers to the food nutrition information in the storage unit 120, and acquires information indicating the amount of each of nutrients corresponding to the identified food. Subsequently, the estimation unit 163 estimates the amount of each of the nutrients indicated by the acquired information as the amount of each of the nutrients corresponding to the identified food.

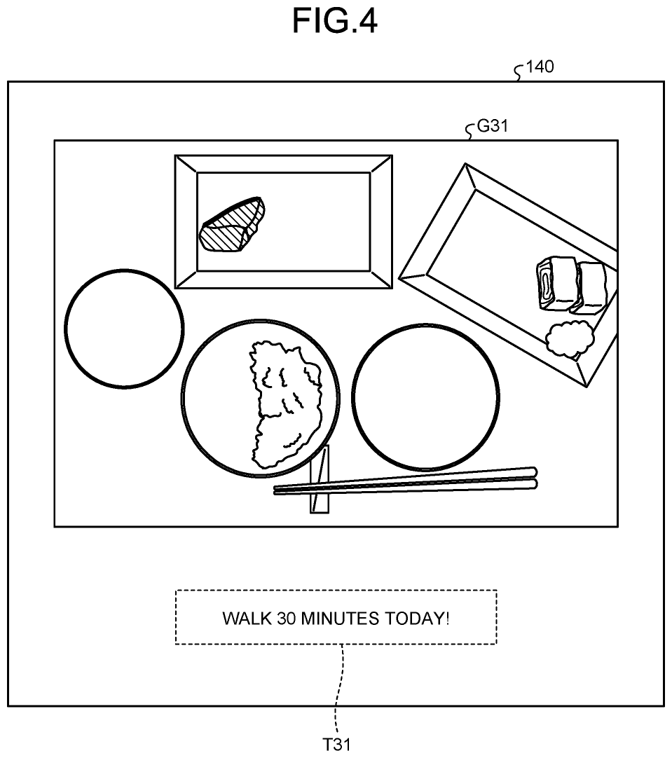

Furthermore, the estimation unit 163 estimates recommended exercise information on an exercise that is recommended to be performed by the target user, on the basis of an after-meal image of the target user. Specifically, if the accepting unit 161 accepts a meal image again from the target user within a predetermined time (for example, within 30 minutes or the like) since a time at which the meal image was accepted, the estimation unit 163 determines that an after-meal image is accepted from the target user. FIG. 4 is a diagram for explaining a process of estimating the recommended exercise information according to the embodiment. In the example illustrated in FIG. 4, the estimation unit 163 determines that an after-meal image G31 is accepted from the target user.

Moreover, the estimation unit 163 estimates, as the recommended exercise information, an exercise time for the exercise that is recommended to be performed by the target user. If the estimation unit 163 determines that the after-meal image is accepted from the target user, the estimation unit 163 estimates an amount of each of the foods taken by the target user on the basis of a comparison between a before-meal image and the after-meal image. Subsequently, the estimation unit 163 estimates calories of each of the foods taken by the target user on the basis of the estimated amount of each of the foods. Then, the estimation unit 163 estimates total calories of the meal taken by the target user by adding the estimated calories of each of the foods. Furthermore, the estimation unit 163 may estimate total calories of meals that are estimated to be taken by the target user in a day, on the basis of the total calories of the meal that has been taken by the target user. Subsequently, if the total calories of meals that are estimated to be taken by the target user exceed calories that need to be taken by the target user in a day, the estimation unit 163 calculates an exercise time corresponding to excessive calories as compared to the calories that need to be taken by the target user.

Moreover, the estimation unit 163 estimates, as the recommended exercise information, a type of the exercise recommended to be performed by the target user. For example, if calories obtained by subtracting the calories that need to be taken by the target user in a day from the total calories of meals that are estimated to be taken by the target user are equal to or larger than a fifth threshold, the estimation unit 163 determines running as a recommendation for the target user. If the estimation unit 163 determines running as the recommendation, the estimation unit 163 calculates an exercise time that is needed to consume the calories that are obtained by subtracting the calories that need to be taken by the target user in a day from the total calories of meals that are estimated to be taken by the target user, on the basis of information indicating calories to be consumed per unit time (for example, 10 minutes) by running.

Furthermore, for example, if the calories obtained by subtracting the calories that need to be taken by the target user in a day from the total calories of meals that are estimated to be taken by the target user are smaller than the fifth threshold, the estimation unit 163 determines walking as a recommendation for the target user. If the estimation unit 163 determines walking as the recommendation, the estimation unit 163 calculates an exercise time that is needed to consume the calories that are obtained by subtracting the calories that need to be taken by the target user in a day from the total calories of meals that are estimated to be taken by the target user, on the basis of information indicating calories to be consumed per unit time (for example, 10 minutes) by walking.

In the example illustrated in FIG. 4, the estimation unit 163 determines walking as the recommendation for the target user. Subsequently, the estimation unit 163 estimates “30 minutes” as the exercise time that is needed to consume the calories that are obtained by subtracting the calories that need to be taken by the target user in a day from the total calories of meals that are estimated to be taken by the target user. If the estimation unit 163 estimates the type of the exercise that is recommended to be performed by the target user and the exercise time, the estimation unit 163 displays information that allows the type of the exercise that is recommended to be performed by the target user and the exercise time to be visually recognized on the screen. For example, the estimation unit 163 displays a character string T31 of “walk 30 minutes today” indicating that 30-minute walking is recommended, at a position located within a predetermined range from a display position of the after-meal image G31.

Meanwhile, the estimation unit 163 acquires, as an exercise that is preferred by the target user, information indicating a type of an exercise (for example, walking, muscle training, or the like) that is input by the target user. Subsequently, the estimation unit 163 may identify the type of the exercise that is input as the exercise preferred by the target user, as the type of the exercise that is recommended to be performed by the target user.

Providing Unit 164

The providing unit 164 provides the recommended food information estimated by the estimation unit 163 to the target user. The providing unit 164 provides the recommended exercise information estimated by the estimation unit 163 to the target user.

2. Flow of Information Processing

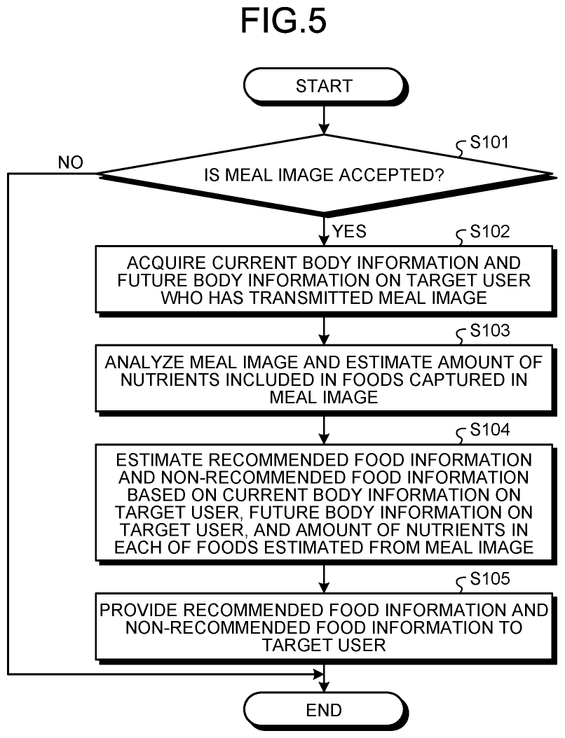

FIG. 5 is a flowchart illustrating the flow of information processing according to the embodiment. As illustrated in FIG. 5, the information processing apparatus 100 determines whether the meal image is accepted (Step S101). If the information processing apparatus 100 determines that the meal image is not accepted (Step S101; No), the process is terminated. In contrast, if the information processing apparatus 100 determines that the meal image is accepted (Step S101; Yes), the information processing apparatus 100 acquires the current body information and the future body information on the target user who has transmitted the meal image (Step S102).

Subsequently, the information processing apparatus 100 analyzes the meal image and estimates an amount of nutrients included in foods captured in the meal image, for each of the foods (Step S103). Subsequently, the information processing apparatus 100 estimates the recommended food information and the non-recommended food information, on the basis of the current body information on the target user, the future body information on the target user, and the amount of the nutrients in each of the foods estimated from the meal image (Step S104). Subsequently, the information processing apparatus 100 provides the recommended food information and the non-recommended food information to the target user (Step S105).

3. Modification

The information processing apparatus 100 according to the embodiment as described above may be embodied in various different modes other than the embodiment as described above. Therefore, other embodiments of the information processing apparatus 100 will be described below. Meanwhile, the same components as those of the embodiment are denoted by the same reference symbols, and explanation thereof will be omitted.

3-1. Estimation of Recommended Menu Information

In the embodiment as described above, the case has been described in which the estimation unit 163 estimates the recommended food information on a recommended food that is recommended to be taken by the target user among the foods captured in the meal image; however, the estimation unit 163 may estimate the recommended food information about other than the recommended food. Specifically, the estimation unit 163 estimates, as the recommended food information, recommended menu information on a recommended menu that is recommended to be taken by the target user among menus provided by a restaurant. Here, the recommended menu information may include non-recommended menu information on a non-recommended menu that is not recommended to be taken by the target user. In other words, the estimation unit 163 may estimate only one of the recommended menu information and the non-recommended menu information, or may estimate both of the recommended menu information and the non-recommended menu information.

For example, the estimation unit 163 acquires, from an external database or the like, information indicating an amount of each of nutrients included in each of menus provided by a restaurant. Subsequently, the estimation unit 163 estimates the recommended menu information and the non-recommended menu information, on the basis of a comparison between information indicating the amount of each of the nutrients included in each of the menus and the amount of each of the nutrients that need to be taken by the target user in a day. The providing unit 164 provides the recommended menu information estimated by the estimation unit 163 to the target user.

3-2. Estimation of Forecast Body Information

Further, the information processing apparatus 100 may estimate forecast body information that is information on a predicted future body of the target user, and provides the forecast body information to the target user. Specifically, the acquisition unit 162 acquires the current body information on the target user, the meal information on a meal that has been taken by the target user, and the exercise information on an exercise that has been performed by the target user. For example, the acquisition unit 162 acquires, via the input unit 130, the current body information, the meal information, and the exercise information that are input by the target user.

The estimation unit 163 estimates the forecast body information that is information on a predicted future body of the target user, on the basis of the current body information, the meal information, and the exercise information that are acquired by the acquisition unit 162. Specifically, if the body information on the user at a predetermined time, the meal information on the meal that has been taken by the user, and the exercise information that has been performed by the user are input, the estimation unit 163 estimates the forecast body information by using a machine learning model M2 that is trained to output information on a body that the user will have after a lapse of a predetermined time period since a predetermined time point. For example, the estimation unit 163 estimates, as the forecast body information, a body shape, weight, a BMI, a body fat percentage, muscle mass, a basal metabolic rate, estimated bone quantity of the target user, or a body shape, a body fat percentage, or muscle mass for each of body parts of the target user. The providing unit 164 provides the forecast body information estimated by the estimation unit 163 to the target user.

Meanwhile, the machine learning models (machine learning model M1 and the machine learning model M2) according to the embodiment and the modification as described above are generated by machine learning using a neural network, such as a convolutional neural network or a recurrent neural network, but are not limited to this example. For example, the machine learning models according to the embodiment and the modification may be generated by using machine learning with a learning algorithm, such as linear regression or logistic regression, instead of the neural network.

4. Effects

As described above, the information processing apparatus 100 according to the embodiment includes the acquisition unit 162, the estimation unit 163, and the providing unit 164. The acquisition unit 162 acquires the current body information that is information on a body of a target user who is a processing target user at a present time and future body information that is information on a body that the target user wants to have after a lapse of a predetermined time since the present time. The estimation unit 163 estimates recommended food information on a food recommended to be taken by the target user among foods that are captured in a meal image obtained by imaging a meal, on the basis of the current body information and the future body information acquired by the acquisition unit 162. The providing unit 164 provides the recommended food information estimated by the estimation unit 163 to the target user.

With this configuration, the information processing apparatus 100 is able to provide the user with the recommended food information that is needed to achieve a goal of changing the current body information on the user to the body information desired by the user. Therefore, the information processing apparatus 100 is appropriately support health management of the user.

Furthermore, the estimation unit 163 estimates the recommended food information including the non-recommended food information on a food that is not recommended to be taken by the target user.

With this configuration, the information processing apparatus 100 is able to provide the user with the non-recommended food information that is needed to achieve the goal of changing the current body information on the user to the body information desired by the user. Therefore, the information processing apparatus 100 is appropriately support the health management of the user.

Moreover, the estimation unit 163 estimates an amount of a nutrient included in a food captured in the meal image, and estimates the recommended food information on the basis of the estimated amount of the nutrient.

With this configuration, the information processing apparatus 100 is able to appropriately estimate the recommended food information on the basis of the amount of the nutrient included in the food captured in the meal image.

Furthermore, the estimation unit 163 estimates, as the recommended food information, an intake amount of a recommended food to be taken by the target user.

With this configuration, the information processing apparatus 100 is able to provide the user with the recommended food information that is needed to achieve the goal of changing the current body information on the user to the body information desired by the user, and that is about the intake amount of the food that is allowed for the user to eat.

Moreover, the information processing apparatus 100 further includes the accepting unit 161. The accepting unit 161 accepts, from the target user, the editing operation on a target user image in which at least a part of a body of the target user is captured. The acquisition unit 162 acquires, as the current body information, a target user image that is not edited through the editing operation accepted by the accepting unit 161, and acquires, as the future body information, a target user image that is edited through the editing operation accepted by the accepting unit 161. The estimation unit 163 estimates the recommended food information on the basis of the target user image that is not edited and the target user image that is edited, where the images are acquired by the acquisition unit 162.

With this configuration, the information processing apparatus 100 allows that target user to easily and visually recognize the body information desired by the target user, so that the target user is able to appropriately acquire the future body information on a target body shape. In addition, the information processing apparatus 100 is able to appropriately estimate the recommended food information on the basis of the appropriate future body information.

Furthermore, the estimation unit 163 estimates, as the recommended food information, the recommended menu information on a menu that is recommended to be taken by the target user among menus provided by a restaurant. The providing unit 164 provides the recommended menu information estimated by the estimation unit 163 to the target user.

With this configuration, the information processing apparatus 100 is able to provide the user with the recommended menu information that is needed to achieve the goal of changing the current body information on the user to the body information desired by the user.

Moreover, the estimation unit 163 estimates the recommended menu information including non-recommended menu information on a menu that is not recommended to be taken by the target user.

With this configuration, the information processing apparatus 100 is able to provide the user with the non-recommended menu information that is needed to achieve the goal of changing the current body information on the user to the body information desired by the user.

Furthermore, the estimation unit 163 estimates the recommended exercise information on an exercise that is recommended to be performed by the target user on the basis of the after-meal image of the target user. The providing unit 164 provides the recommended exercise information estimated by the estimation unit 163 to the target user.

With this configuration, the information processing apparatus 100 is able to provide the user with the recommended exercise information that is needed to achieve the goal of changing the current body information on the user to the body information desired by the user. Therefore, the information processing apparatus 100 is able to appropriately support the health management of the user.

Moreover, the estimation unit 163 estimates, as the recommended exercise information, an exercise time of an exercise that is recommended to be performed by the target user.

With this configuration, the information processing apparatus 100 is able to provide the user with the recommended exercise information that is needed to achieve the goal of changing the current body information on the user to the body information desired by the user, and that is information on the exercise time recommended for the user.

Furthermore, the estimation unit 163 estimates, as the recommended exercise information, a type of an exercise that is recommended to be performed by the target user.

With this configuration, the information processing apparatus 100 is able to provide the user with the recommended exercise information that is needed to achieve the goal of changing the current body information on the user to the body information desired by the user, and that is information on the type of the exercise recommended for the user.

Moreover, the acquisition unit 162 further acquires the meal information on a meal that has been taken by the target user and the exercise information on an exercise that has been performed by the target user. The estimation unit 163 estimates the forecast body information that is information on a predicted future body of the target user, on the basis of the meal information and the exercise information acquired by the acquisition unit 162. The providing unit 164 provides the forecast body information estimated by the estimation unit 163 to the target user.

With this configuration, the information processing apparatus 100 is able to provide the forecast body information to the user, so that it is possible to raise awareness of the health management of the target user.

Furthermore, if the body information on the user at a predetermined time, the meal information on the meal that has been taken by the user, and the exercise information on the exercise that has been performed by the user are input, the estimation unit 163 estimates the forecast body information by using a machine learning model that is trained to output information on a body that the user will have after a lapse of a predetermined time since a predetermined time point.

With this configuration, the information processing apparatus 100 is able to appropriately estimate the forecast body information by using the machine learning model.

Moreover, the estimation unit 163 estimates, as the forecast body information, a body shape, weight, chest circumference, waist circumference, hip circumference, a body mass index (BMI), a body fat percentage, muscle mass, a basal metabolic rate, or estimated bone quantity of the target user, or a body shape, a body fat percentage, or muscle mass of each of body parts of the target user.

With this configuration, the information processing apparatus 100 is able to estimate various kinds of forecast body information.

5. Hardware Configuration

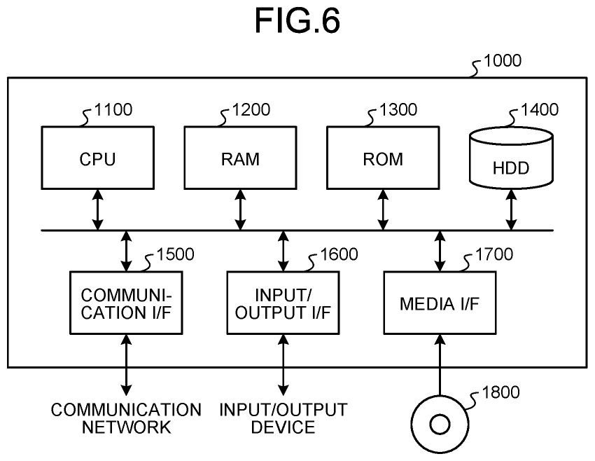

The information processing apparatus 100 according to the embodiment as described above is implemented by, for example, a computer 1000 configured as illustrated in FIG. 6. FIG. 6 is a hardware configuration diagram illustrating an example of a computer that implements the functions of the information processing apparatus 100. The computer 1000 includes a CPU 1100, a RAM 1200, a ROM 1300, an HDD 1400, a communication interface (I/F) 1500, an input/output I/F 1600, and a media I/F 1700.

The CPU 1100 operates based on a program stored in the ROM 1300 or the HDD 1400, and controls each of the units. The ROM 1300 stores therein a boot program that is executed by the CPU 1100 at the time of activation of the computer 1000, a program that depends on the hardware of the computer 1000, or the like.

The HDD 1400 stores therein a program executed by the CPU 1100, data used by the program, and the like. The communication I/F 1500 receives data from other apparatuses via a predetermined communication network, sends the data to the CPU 1100, and transmits data generated by the CPU 1100 to the other apparatuses via the predetermined communication network.

The CPU 1100 controls an output device, such as a display or a printer, and an input device, such as a keyboard or a mouse, via the input/output I/F 1600. The CPU 1100 acquires data from the input device via the input/output I/F 1600. Further, the CPU 1100 outputs the generated data to the output device via the input/output I/F 1600.

The media I/F 1700 reads a program or data stored in a recording medium 1800, and provides the program or the data to the CPU 1100 via the RAM 1200. The CPU 1100 loads the program from the recording medium 1800 to the RAM 1200 via the media I/F 1700, and executes the loaded program. Examples of the recording medium 1800 include an optical recording medium, such as a digital versatile disk (DVD) or a phase change rewritable disk (PD), a magneto-optical recording medium, such as a magneto-optical disk (MO), a tape medium, a magnetic recording medium, and a semiconductor memory.

For example, if the computer 1000 functions as the information processing apparatus 100 according to the embodiment, the CPU 1100 of the computer 1000 executes a program loaded on the RAM 1200, and implements the functions of the control unit 160. The CPU 1100 of the computer 1000 reads the program from the recording medium 1800 and executes the program; however, as another example, it may be possible to acquire the program from a different apparatus via a predetermined communication network.

Thus, some embodiments of the present application have been described in detail above based on the drawings, but the embodiments are mere examples, and the present invention may be embodied in different modes with various changes and modifications based on knowledge of a person skilled in the art, in addition to the modes described in the detailed description of the preferred embodiments in this application.

6. Others

Of the processes described in the embodiments and the modifications, all or part of a process described as being performed automatically may also be performed manually. Alternatively, all or part of a process described as being performed manually may also be performed automatically by known methods. In addition, the processing procedures, specific names, and information including various kinds of data and parameters illustrated in the above-described document and drawings may be arbitrarily changed unless otherwise specified. For example, various kinds of information illustrated in each of the drawings are not limited to the information illustrated in the drawings.

The components illustrated in the drawings are functionally conceptual and do not necessarily have to be physically configured in the manner illustrated in the drawings. In other words, specific forms of distribution and integration of the apparatuses are not limited to those illustrated in the drawings, and all or part of the apparatuses may be functionally or physically distributed or integrated in arbitrary units depending on various loads or use conditions.

Furthermore, the information processing apparatus 100 as described above may be implemented by a plurality of computers, and a configuration may be flexibly changed such that some functions may be implemented by calling an external platform or the like by an application programming interface (API), network computing, or the like.

Moreover, the embodiments and the modifications as described above may be appropriately combined as long as the processes do not conflict with each other.

According to one aspect of the embodiment, it is possible to appropriately support health management of a user.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A non-transitory computer-readable recording medium with an information processing program stored thereon, wherein the program instructs a computer to execute:
acquiring current body information that is information on a body of a target user who is a processing target user at a present time and future body information that is information on a body that the target user wants to have after a lapse of a predetermined time since the present time;
estimating recommended food information on a food recommended to be taken by the target user among foods that are captured in a meal image obtained by imaging a meal, on the basis of the current body information and the future body information acquired at the acquiring; and
providing the recommended food information estimated at the estimating to the target user.

2. The computer-readable recording medium according to claim 1, wherein the estimating includes estimating the recommended food information including non-recommended food information on a food that is not recommended to be taken by the target user.

3. The computer-readable recording medium according to claim 1, wherein the estimating includes estimating an amount of a nutrient included in a food captured in the meal image, and estimating the recommended food information on the basis of the estimated amount of the nutrient.

4. The computer-readable recording medium according to claim 1, wherein the estimating includes estimating, as the recommended food information, an intake amount of a recommended food to be taken by the target user.

5. The computer-readable recording medium according to claim 1, further comprising:
accepting, from the target user, editing operation on a target user image in which at least a part of a body of the target user is captured, wherein
the acquiring includes acquiring, as the current body information, a target user image that is not edited through the editing operation accepted at the accepting, and acquires, as the future body information, a target user image that is edited through the editing operation accepted at the accepting, and
the estimating includes estimating the recommended food information on the basis of the target user image that is not edited and the target user image that is edited, the target user images being acquired at the acquiring.

6. The computer-readable recording medium according to claim 1, wherein
the estimating includes estimating, as the recommended food information, recommended menu information on a menu that is recommended to be taken by the target user among menus provided by a restaurant, and
the providing includes providing the recommended menu information estimated at the estimating to the target user.

7. The computer-readable recording medium according to claim 6, wherein the estimating includes estimating the recommended menu information including non-recommended menu information on a menu that is not recommended to be taken by the target user.

8. The computer-readable recording medium according to claim 1, wherein
the estimating includes estimating recommended exercise information on an exercise that is recommended to be performed by the target user on the basis of an after-meal image of the target user, and
the providing includes providing the recommended exercise information estimated at the estimating to the target user.

9. The computer-readable recording medium according to claim 8, wherein the estimating includes estimating, as the recommended exercise information, an exercise time of an exercise that is recommended to be performed by the target user.

10. The computer-readable recording medium according to claim 8, wherein the estimating includes estimating, as the recommended exercise information, a type of an exercise that is recommended to be performed by the target user.

11. The computer-readable recording medium according to claim 1, wherein
the acquiring includes acquiring meal information on a meal that has been taken by the target user and exercise information on an exercise that has been performed by the target user,
the estimating includes estimating forecast body information that is information on a predicted future body of the target user, on the basis of the meal information and the exercise information acquired at the acquiring, and
the providing includes providing the forecast body information estimated at the estimating to the target user.

12. The computer-readable recording medium according to claim 11, wherein the estimating includes estimating, if the body information on the user at a predetermined time, the meal information on the meal that has been taken by the user, and the exercise information on the exercise that has been performed by the user are input, the forecast body information by using a machine learning model that is trained to output information on a body that the user will have after a lapse of a predetermined time period since a predetermined time point.

13. The computer-readable recording medium according to claim 11, wherein the estimating includes estimating, as the forecast body information, one of a body shape, weight, chest circumference, waist circumference, hip circumference, a body mass index (BMI), a body fat percentage, muscle mass, a basal metabolic rate, and estimated bone quantity of the target user, and a body shape, a body fat percentage, or muscle mass of each of body parts of the target user.

14. An information processing apparatus comprising:
an acquisition unit that acquires current body information that is information on a body of a target user who is a processing target user at a present time and future body information that is information on a body that the target user wants to have after a lapse of a predetermined time since the present time;
an estimation unit that estimates food information on a food recommended to be taken by the target user among foods that are captured in a meal image obtained by imaging a meal, on the basis of the current body information and the future body information acquired by the acquisition unit; and
a providing unit that provides the recommended food information estimated by the estimation unit to the target user.

15. An information processing method comprising:
acquiring current body information that is information on a body of a target user who is a processing target user at a present time and future body information that is information on a body that the target user wants to have after a lapse of a predetermined time since the present time;
estimating recommended food information on a food recommended to be taken by the target user among foods that are captured in a meal image obtained by imaging a meal, on the basis of the current body information and the future body information acquired at the acquiring; and
providing the recommended food information estimated at the estimating to the target user.
